(19) United States
(12) Patent Application Publication
(10) Pub. No.: US 2008/0043903 A1
(43) Pub. Date: Feb. 21, 2008

(54) Image-Guided Intensity-Modulated X-Ray Brachytherapy System

(76) Inventors: Fang-Fang Yin, Chapel Hill, NC (US); Jae Ho Kim, West Bloomfield, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/628,935

(22) PCT Filed: Jun. 7, 2005
(86) PCT No.: PCT/US05/19984

§ 371 Date: Dec. 7, 2006

Related U.S. Application Data
(60) Provisional application No. 60/577,677, filed on Jun. 7, 2004.

(51) Int. Cl. A61B 6/00

(52) U.S. Cl. 378/21; 378/65

(57) ABSTRACT

In some embodiments, without limitation, the invention comprises a modulated image-guided x-ray brachytherapy system including a source providing low-energy photons, the source configured for placement at least partially within a patient, a control system configured to modulate the source, and an imaging systems for locating the source relative to a treatment location in the patient. The system may further comprises a target at least partially within the patient, a generator located outside the patient for providing low-energy photons, the generator configured to direct low-energy photons to the target, and a conduit between the generator and the target providing a path for the low-energy photons to travel inside the patient. The system may further comprise an in situ x-ray generator for insertion within the patient. The system may further comprise a radioactive pellet.

### CROSS-REFERENCE TO RELATED APPLICATIONS

This application claims priority based on U.S. Provisional Patent Application No. 60/577,677 filed Jun. 7, 2004 which is hereby incorporated by reference in full.

### FIELD OF THE INVENTION

Generally, the present invention relates to the use of an x-ray system for cancer treatment. More specifically, the present invention relates to the use of an x-ray system with an in situ x-ray source for treating tumors.

### BACKGROUND OF THE INVENTION

In the field of medicine, nuclear radiation may be used for diagnostic and therapeutic treatment of patients inflicted with cancer. Typically, more than half of these patients need radiation therapy either as a primary or an adjunct mode of treatment. Conventional medical radiation sources used in these treatments include large fixed-position machines such as linear accelerators, as well as small, transportable radiation-generating probes that provide a boost therapy. In the latter treatment system, miniaturized probes capable of producing a high dose of radiation in a pre-defined geometry are inserted into a treatment volume. The treatment is commonly referred to as brachytherapy because the radiation source is located close to or, in some cases, within, the treatment volume.

The advantage of brachytherapy is that very high doses of ionizing radiation can be delivered to a localized volume of tissue such that the radiation is supplied primarily to the treatment volume without significantly affecting tissues in adjacent volumes. This ability, when combined with a rapid reduction in the radiation dose as a function of distance, shields distant anatomies from spurious radiation. Hence, the technique has provided excellent results for localized control of various tumors.

In applications where tumors under treatment are in the patient's prostate gland, an applicator such as a perineal template is commonly employed with one or more probes that contain seeds or radiation sources. The template has an array of openings for accepting a plurality of sequential tandem brachytherapy probes or needles. During operation, the template is positioned near tumors to be treated and referenced to one or more scanned images before the seeds are inserted into the openings. For anatomical regions where there is no body cavity, the interstitial implantation of radioactive needles is preferred. The needles are typically long and hollow with small outer diameter and at least one sharp end to allow penetration through the tissue. Small gauge needles may deflect as they encounter obstructions such as calcification or changes in tissue impedance because such needles may be easily bowed.

Known low power miniaturized x-ray sources are implantable into a patient body for direct delivery of x-ray radiation. However, these miniaturized sources have disadvantages. The tube has very limited power because the x-ray source should be small enough to be implanted into the body. For treatment of tumors, for example, brain tumors, a high dose rate for single dose irradiation is generally preferred. Hence, this tube may not provide sufficient radiation as required for treatment.

Additionally, high vacuum x-ray sources with high voltage (up to 90 kV) are difficult to introduce into a human body. These sources also produce an x-ray energy continuum, known as bremsstrahlung, which may destroy healthy tissue in addition to the predefined “useful” x-ray energy. Further, during relatively long exposures, typically several minutes, the temperature of the anode that is set on the end of implantable x-ray source may rise significantly.

Further, the x-ray device may sometimes be used in environments in which there are low-level dc and ac electromagnetic fields (due to electric power, the field of the earth, etc.) that are capable of deflecting the electron beam from the anode of implantable x-ray source. Special measures therefore need to be taken against the influences of electric fields on electron beam. There is also a large diameter opening for the implantable x-ray source, typically 5 to 7 mm.

Alternatively, a radioactive seed, or pellet, may be used to deliver radiation to a treatment location. During a brachytherapy operation, a physician needs to know the exact position of the seeds, as well as the radiation dosage distribution from these seeds. It is also desirable to quantify the radiation received by the surrounding organs. Images of the treatment area are obtained from modalities such as x-ray radiograph, computed tomography, magnetic resonance, ultrasound, or nuclear medicine scans of the patient during a treatment simulation procedure. The information obtained from the images is correlated with the position of the template or the needles for intracavity or interstitial treatment. The position of the seed inside the patient is determined relative to the needle and the template as a function of the needle length and orientation, minus a length of a remaining needle portion outside the template.

A dosimetry program may be configured to compute the radiation dose by taking into consideration the intensity of the radioactive sources and their coordinates relative to the tumor. Needle bowing and deflection introduce errors in the coordinates of the radioactive seeds and result in miscalculation of the radiation dose. Implications of the error in the radiation dose calculation include underexposure of the tumor and exposure of normal tissue to harmful radiation.

In addition, bending of the needle may result in breaking of the needle in the tissue. This may cause tissue damage and may require surgery to remove the broken needle pieces.

It would therefore be useful to develop an x-ray device that can overcome the obstacles set forth above.

### SUMMARY

In some embodiments, without limitation, the invention comprises a modulated image-guided x-ray brachytherapy system including a source providing low-energy photons, the source configured for placement at least partially within a patient, a control system configured to modulate the source, and an imaging system for locating the source relative to a treatment location in the patient. The system may further comprise a target at least partially within the patient, a generator located outside the patient for providing low-energy photons, the generator configured to direct low-energy photons to the target, and a conduit between the generator and the target providing a path for the low-energy photons to travel inside the patient. The system may further comprise an in situ x-ray generator for insertion within the patient. The system may further comprise a radioactive pellet.

In some embodiments, without limitation, the system may further comprise a mount engaged with the conduit and the target, the mount surrounding the target, and at least one opening disposed upon the mount, whereby said at least one opening provides a path for the low-energy photons to interact with the patient. The system may further comprise a shutter for selectively obstructing the at least one opening, the control system selectively positioning said shutter relative to said opening.

In some embodiments, without limitation, the invention comprises an image-guided intensity-modulated x-ray brachytherapy system comprising a source providing low-energy photons for treatment of a patient, the source configured for placement at least partially within a patient, a control element for modulating the intensity of the source, a detector sensitive to the low-energy photons, and a processor operatively coupled to said detector and said control element, said processor producing an image of a treatment location. The processor may be operatively coupled to said source.

In some embodiments, an image-guided intensity-modulated x-ray brachytherapy system is provided comprising a rotable x-ray source configured for placement at least partially within a patient, an x-ray detector having a plurality of sensing elements for detecting x-rays that have passed through the patient, and a processor operatively connected to said x-ray detector for processing a plurality of output images from said x-ray detector to produce a tomographic image, whereby the tomographic image is used for at least one of a three dimensional visualization of a treatment location, treatment localization, treatment analysis, and treatment verification. The system may further comprise an external x-ray generator, said external x-ray generator positioned outside said patient, and wherein said rotable x-ray source comprises a target for redirecting the x-rays provided by said external x-ray generator. The system may be configured such that the rotable x-ray source comprises an in situ x-ray generator. The system may be configured such that the rotable x-ray source comprises a radioactive pellet.

In some embodiments, an image-guided intensity-modulated x-raybrachytherapy system is provided comprising a rotable x-ray source configured for in situ treatment within a patient, an x-ray detector having a plurality of sensing elements and producing an output, and a processor operatively connected to said x-ray detector for storing a plurality of outputs, said processor constructing a three dimensional representation of a treatment location within the patient.

In some embodiments, without limitation, the invention comprises a system for image-guided intensity-modulated x-ray brachytherapy comprising at least one x-ray source configured for placement at least partially within a patient, a control system modulating said at least one x-ray source, a data processor operatively connected to said control system, wherein said data processor determines a strategy for at least one x-ray source location and at least one x-ray source intensity for treating at least one treatment location, and wherein said data processor communicates said strategy to said control system. The system may further comprise an optimizing algorithm for improving the efficiency of locating the at least one source and modulating the at least one x-ray source.

In some embodiments, the invention comprises a planning method for image-guided intensity-modulated x-ray brachytherapy comprising the steps of generating x-rays from at least one in situ x-ray source, imaging a treatment location, generating a treatment plan, and modulating said at least one in situ x-ray source according to said treatment plan. The method may further provide that the imaging is a tomographic image.

In some embodiments, a system for controlling an image-guided intensity-modulated x-ray brachytherapy system is provided comprising an in situ x-ray source located at least partially within a patient for treating a treatment location, a modulator selectively controlling said in situ x-ray source intensity, a positioner for locating said in situ x-ray source within a patient, an imaging system for collecting real-time treatment information, and a data processor operatively connected to said imaging system, said positioner, and said modulator, said data processor following a treatment plan, said data processor validating said treatment at said treatment location with said real-time treatment information, such that said data processor communicates with said positioner and said modulator to realize said treatment plan.

The invention may further provide that the modulator controls at least one of a source energy level, a source density, a source shape, and a source angle of emission. The system may further provide that locating said in situ x-ray source comprises at least one of a rotation angle and a depth of penetration within said patient. The system may further comprise a heating element located at least partially within a patient for treating the treatment location.

In some embodiments, without limitation, the invention comprises a method for controlling an image-guided intensity-modulated x-ray brachytherapy system comprising the steps of imaging of a treatment location, determining a method of treatment, planning a radiative treatment cycle, said planning including an inverse treatment method to determine optimal exposures for the treatment location, outputting a treatment plan, and controlling a brachytherapy device using said treatment plan. The method may further provide that the radiative treatment cycle comprises at least one component of time, location, radiative shape, and radiative type.

In some embodiments, an image-guided intensity-modulated x-ray brachytherapy system is provided comprising a probe configured for insertion at least partially within a patient, an x-ray source configured for placement at least partially within a patient, and an adjunctive therapy for treating the patient. The system may further provide that the adjunctive therapy comprises heating a portion of the probe.

### BRIEF DESCRIPTION OF THE DRAWINGS

The present invention will now be described, by way of example only and without limitation, with reference to the accompanying drawings, in which:

FIG. 1 is a cross-sectional view of a modulated image-guided x-ray brachytherapy system according to the present invention;

FIG. 2 is a cross-sectional view of an external source modulated image-guided x-ray brachytherapy system according to the embodiment of FIG. 1;

FIG. 3 is a cross-sectional view of an embodiment of a metallic target needle probe for use with the system of FIG. 2;

FIG. 4 is a cross-sectional view of an embodiment of a crystalline target needle probe for use with the system of FIG. 2;

FIG. 5 is a cross-sectional view of an embodiment of a needle probe having a heating element for use with the system of FIG. 2;

FIG. 6 is a cross-sectional view of an embodiment of a miniature in situ x-ray generator for use with the system of FIG. 1;

FIG. 7 is a simplified layout of an embodiment for a control system for the miniature in situ x-ray generator of FIG. 6.

FIG. 8 is a cross-sectional view of a needle probe and a radioactive pellet for use with the system of FIG. 1;

FIG. 9 is a cross-sectional view of a needle probe and an alternate embodiment for a radioactive pellet for use with the system of FIG. 1;

FIG. 10A is an external top view of a single-window needle probe for use with the system of FIG. 1;

FIG. 10B is a radial cross-sectional view of a single-window needle probe for use with the system of FIG. 1;

FIG. 10C is a cross-sectional view of a single-window needle probe for use with the system of FIG. 1;

FIG. 11A is an external top view of a multi-window needle probe for use with the system of FIG. 1;

FIG. 11B is a radial cross-sectional view of a multi-window needle probe for use with the system of FIG. 1;

FIG. 11C is a cross-sectional view of a multi-window needle probe for use with the system of FIG. 1;

FIG. 12 is a cross-sectional view of an axial modulator, or shutter, for use with the windowed needles of FIGS. 10 and 11;

FIG. 13 is a radial cross-sectional view of a radial modulator, or shutter, for use with the windowed needles of FIGS. 10 and 11;

FIG. 14A is a top view of a slot-type modulator, or shutter, for use with the windowed needles of FIGS. 10 and 11;

FIG. 14B is a cross-sectional view of a slot-type modulator, or shutter, for use with the windowed needles of FIGS. 10 and 11;

FIG. 15 is a simplified layout of a processor for use with the system of FIG. 1;

FIG. 16 is a simplified layout of a modulated image-guided x-ray brachytherapy system according to the present invention;

FIG. 17A is a side view of an in situ source in a first position and a first x-ray energy for use with the system of FIG. 16;

FIG. 17B is a side view of an in situ source in a second position and a first x-ray energy for use with the system of FIG. 16;

FIG. 17C is a side view of an in situ source in a first position and a second x-ray energy for use with the system of FIG. 16;

FIG. 18 is a simplified layout of a multiple source, modulated, image-guided x-ray brachytherapy system according to the present invention;

FIG. 19 is a process flow for the image-guidance for use with the systems of FIGS. 16 and 18;

FIG. 20 is a process flow for fluoroscopic monitoring of the treatment process for use with the systems of FIGS. 16 and 18;

FIG. 21 is a process flow for verifying and documenting treatment for use with the systems of FIGS. 16 and 18;

FIG. 22 is a process flow for tomographic imaging for use with the systems of FIGS. 16 and 18;

FIG. 23 is a process flow for a control system for the x-ray source for use with the systems of FIGS. 16 and 18;

FIG. 24 is a process flow for controlling the intensity and time of exposure for the x-ray source for use with the systems of FIGS. 16 and 18;

FIG. 25 is a process flow for controlling a mechanical x-ray modulating system for use with the systems of FIGS. 16 and 18;

FIG. 26 is a process flow for controlling an adjunctive treatment method for use with the systems of FIGS. 16 and 18;

FIG. 27 is a process flow for comparing planned treatment mapping and recorded treatment mapping for use with the systems of FIGS. 16 and 18; and

FIG. 28 is a process flow for a treatment planning and optimization method for use with the systems of FIGS. 16 and 18.

### DETAILED DESCRIPTION

Generally, the present invention comprises methods and apparatus for more accurately and effectively administering radiation treatments to and/or for imaging biological tissue.

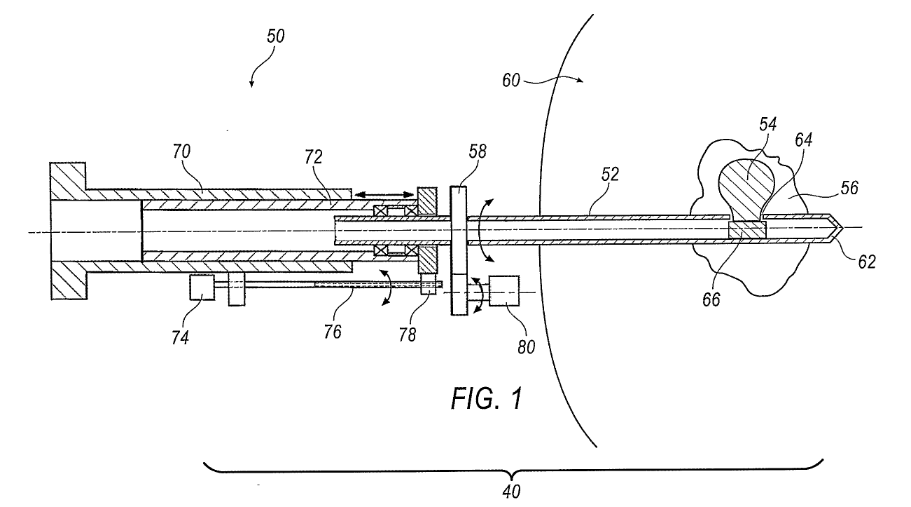

Referring to FIG. 1, in some embodiments, without limitation, the invention comprises a system 40 including a positioning apparatus 50 locating an x-ray source 66 for the delivery of x-rays 54 to a treatment location 56, where treatment location 56 is generally a tumor. X-ray source 66 is generally disposed within a needle 52. Needle 52 is rotably attached to positioning apparatus 50 by a rotating hub 58. Needle 52 is inserted into patient 60 with the assistance of a pointed tip 62 located at the distal end of needle 52. A window 64 is provided through needle 52 allowing x-rays 54 produced by x-ray source 66 to exit needle 52 and interact with treatment location 56.

Positioning apparatus 50 comprises an outer tube 70 and an inner tube 72. Inner tube 72 slides within outer tube 70 and precise positioning of the extension of inner tube 72 is provided by an axial motor 74, a threaded screw 76 and a nut 78. Nut 78 is attached to inner tube 72 and threadingly engages threaded screw 76. Axial motor 74 is attached to outer tube 70 and turns threaded screw 76. As axial motor 74 turns screw 76, nut 78 rides along screw 76 and thus extends or retracts inner tube 72 relative to outer tube 70 depending upon the direction of rotation of screw 76. A rotary motor 80 gearingly engages rotating hub 58 and provides rotation to needle 52. Thus, for treatment, window 64 is provided in situ to treatment location 56 where positioning apparatus 50 allows for precise location of window 64 axially and radially.

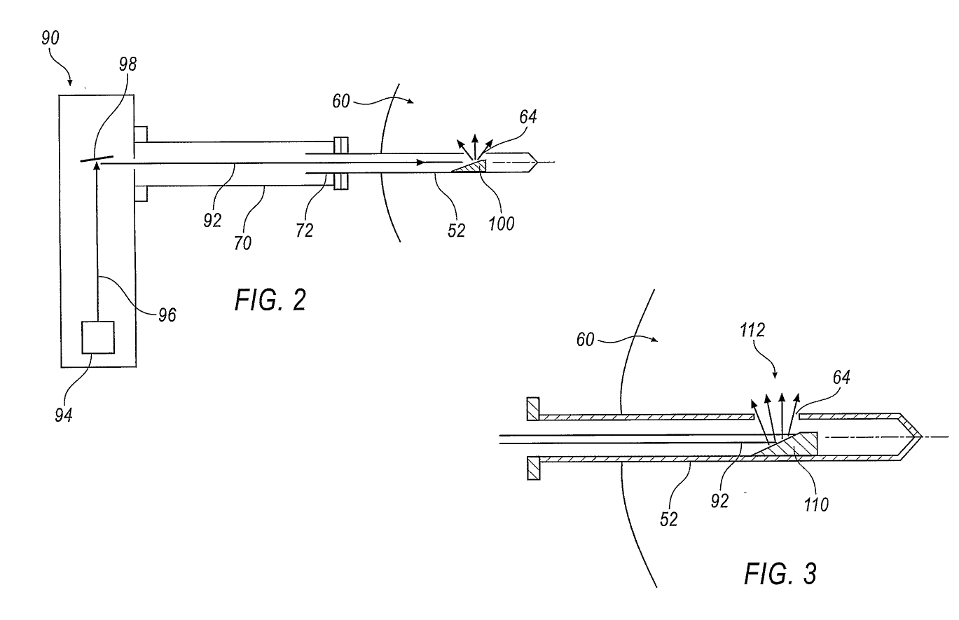

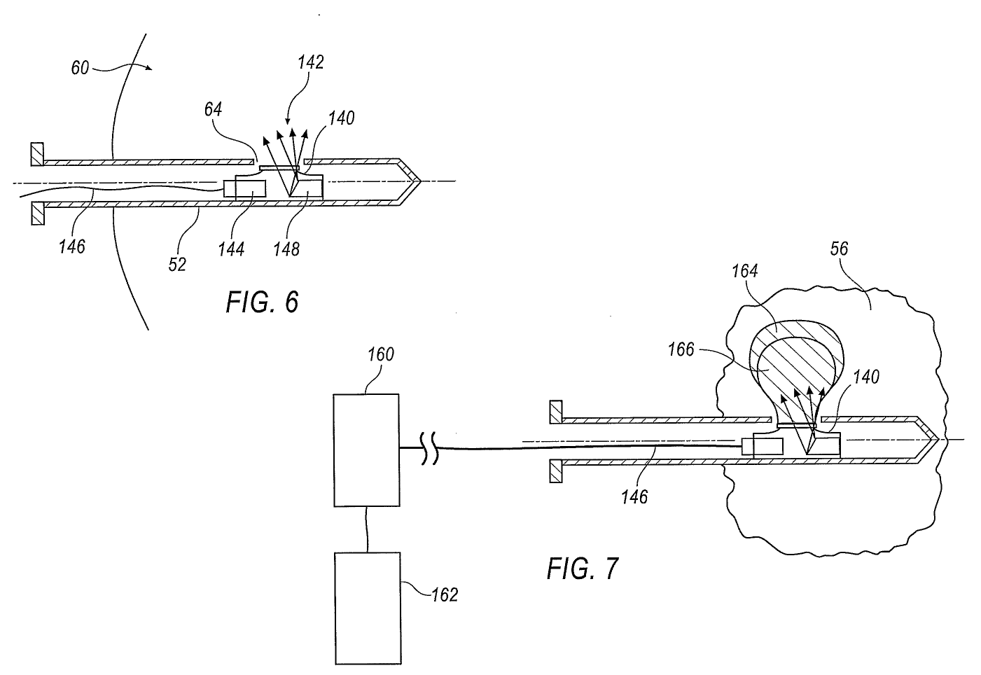

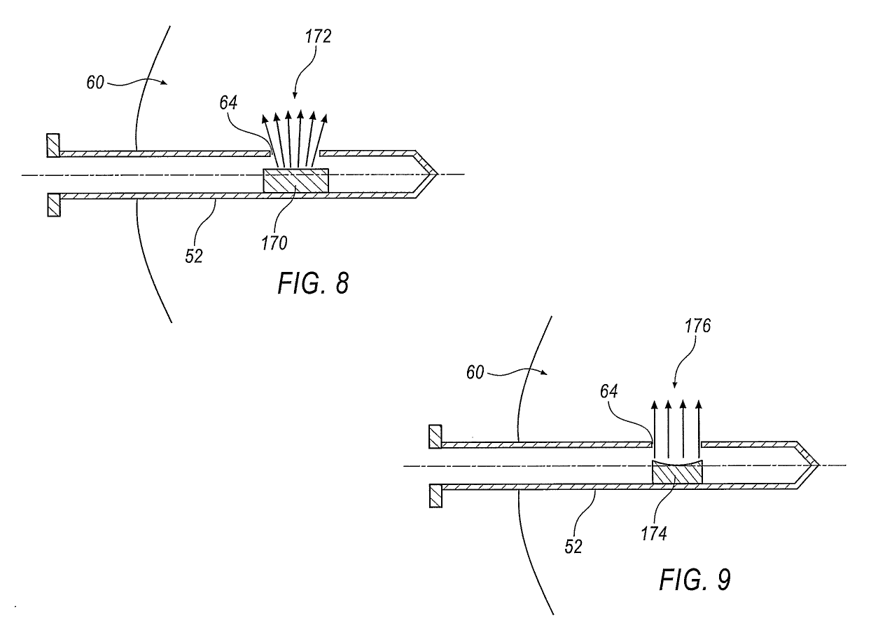

X-ray source 66 as used herein comprises, but is not limited to, x-ray devices capable of providing x-ray treatments, devices capable of altering the shape of the x-rays, devices capable to imaging tissue, and devices capable of forming tomographic images. These devices may include externally generated x-rays that are directed to x-ray source 66 (explained in detail with respect to FIG. 2), in situ generated x-rays (explained in detail with respect to FIG. 6), or radiative materials such as a radioactive pellet (explained in detail with respect to FIG. 8). Examples of such sources are explained in detail below. However, other x-ray devices may be used without departing from the spirit of the present invention. Without limiting the scope of the invention, the device is preferably a needle, a probe, or similar type device that is configured to emit low-energy photon beams. Certain of such devices are known to those of ordinary skill in the art, as examples only and without limitation, those disclosed in U.S. Pat. No. 6,493,421 and U.S. Pat. No. 6,580,940, each of which is incorporated fully herein.

Referring to FIG. 2, an external generator 90 is shown providing x-rays 92 to needle 52. An electron gun 94 emits high energy electrons 96 that strike an anode 98. As high energy electrons 96 strike anode 98, x-rays 92 are emitted. X-rays 92 then travel through outer tube 70, inner tube 72 and needle 52 and ultimately strike target 100. As x-rays 92 strike target 100, x-rays 92 are redirected through window 64 and interact with patient 60. In another embodiment, target 100 behaves as x-ray source 66 illustrated in FIG. 1.

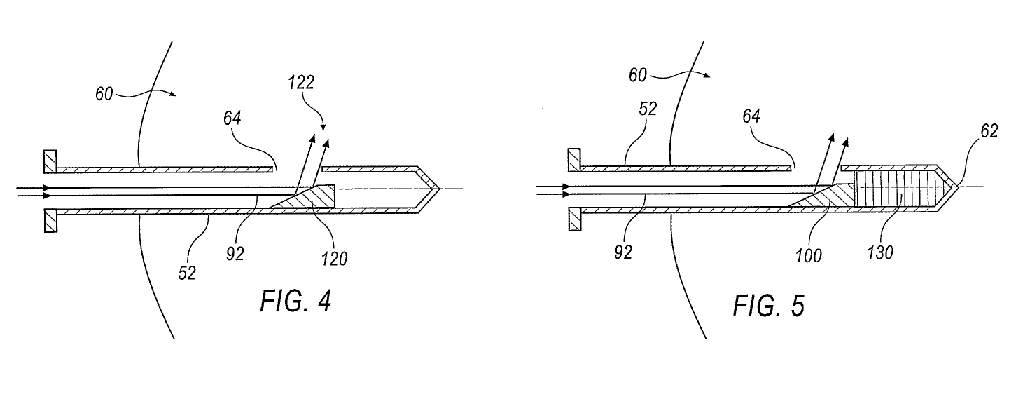

Referring generally to FIGS. 3 and 4, some different materials for target 100 are illustrated. Without limiting the scope of usable target materials, a detailed description of exemplary target 100 materials is found in U.S. Pat. No. 6,580,940. FIG. 3 illustrates the use of a metallic target 110. As x-rays 92 interact with metallic target 110, a wide dispersion pattern of x-rays 112 results when exiting window 64. FIG. 4 illustrates the use of a crystalline target 120. As x-rays 92 interact with crystalline target 120, a narrow dispersion pattern of x-rays 122 results when exiting window 64.

Referring to FIG. 5, a heating element 130 is illustrated as an adjunctive treatment to the x-ray therapy. In some embodiments, without limitation, heating element 130 is located near tip 62 of needle 52. Heating element 130 may be in contact with target 100 so that heat is transferred from target 100 to heating element 130 as target 100 naturally becomes hot from interacting with x-rays 92. Such a dual device thus is comprised of a heating element 130 and x-ray delivery device, illustrated here as target 100, and may be used to deliver heat and x-ray sources in situ to treatment location 56. Thus, in some embodiments, without limitation, the invention may deliver both heat and x-rays simultaneously to the treatment region to maximize the biological affect of cancer treatment.

Although in some embodiments, heating element 130 is illustrated as a passive component, other embodiments are within the scope of the invention. As additional examples only, heating element 130 may be attached to the insertable needle or may be the needle itself. Additionally, heating element 130 may be an active component that is self-heating, or may be a separate instrument. The heating device may be any heating device known to those of skill in the art to be applicable as a therapy and that may be usable in conjunction with radiation therapy.

Referring to FIG. 6, an in situ x-ray generator 140 is shown. Although not illustrated, needle 54 is used in conjunction with positing apparatus 50 (see FIG. 1). In some embodiments, x-rays 142 are generated within needle 52 and patient 60 using a miniature in situ x-ray generator 140. An electron gun 144 is supplied with high voltage by a control line 146. When active, electrons are generated by an electron gun 144 and strike an anode 148 producing x-rays 142. X-rays 142 then leave needle 52 through window 64 in situ x-ray generator 140.

Referring to FIG. 7, a simplified control system for in situ x-ray generator 140 is shown. When treating patients, modulation of in situ x-ray generator 140 is desirable. Depending upon the nature of treatment location 56, production of a high intensity 164 or a low intensity 166 x-ray source may be desired. In either case, in situ x-ray generator 140 may be modulated to provide various intensities as desired. A high voltage modulator 160 provides power to in situ x-ray generator 140 and may also include a feedback mechanism to control the x-ray output. Further, processor 162 may command high voltage modulator 160 to provide user determinable x-ray output based on an algorithmic decision or user inputs. Additionally, processor 162 maybe used to control the energy output and duration of x-rays produced by external generator 90 of FIG. 2.

In some embodiments, without limitation, referring to FIG. 8, a radiative material 170, also described as a radioactive pellet, is illustrated as source 66. Radiative material 170 may be positioned within needle 52 near window 64 such that x-rays 172 may interact with patient 60.

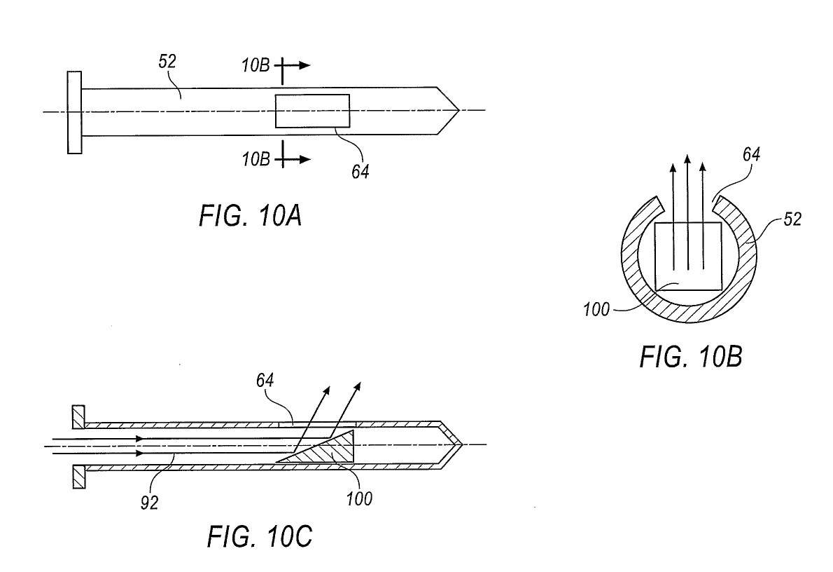

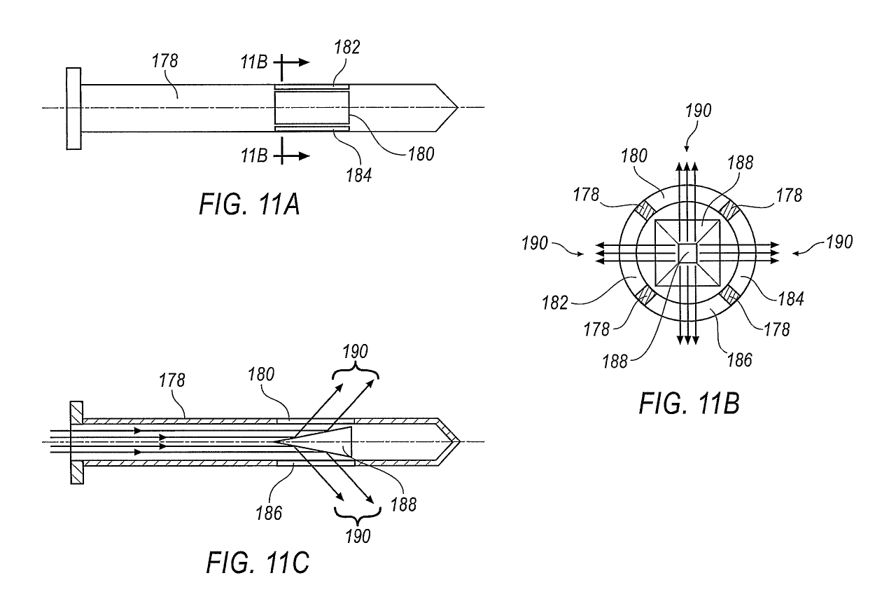

Referring to FIGS. 10A-10C, a single window needle 52 is shown. Window 64 is an opening in needle 52 that provides access for x-rays 92 to exit the lumen of needle 52. In some embodiments, referring to FIGS. 11A-11C, a multiple window needle 178 is shown. A first, second, third, and fourth window 180, 182, 184, and 186 respectively are shown. Multiple window needle 178 comprises a pyramidal target 188 for deflecting x-rays 92 outside the lumen of multiple window needle 178. Thus, treatment at treatment location 56 is provided in all directions based on the dispersion pattern of x-rays 190.

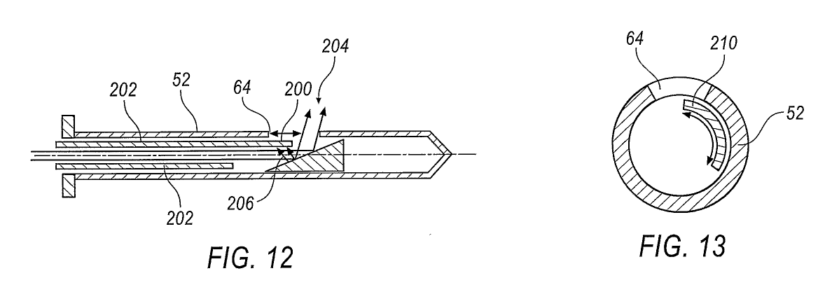

During patient treatment, modulation of the produced x-rays is preferred in order to control the direction and dosage provided to the patient. Further, without limiting the scope of the invention, modulation of the x-rays may be required for various imaging modalities. Referring to FIG. 12, an axial modulator 200 is shown. Axial modulator 200 is attached to, or is part of, a control cannula 202. Control cannula 202 slides within needle 52 and is controlled at the proximal end of needle 52 by a linear actuator as known by those skilled in the art. As control cannula 202 slides within needle 52, axial modulator 200 modifies the geometry of window 64. Without axial modulator 200, a wide dispersion pattern of x-rays results as is illustrated in FIG. 3. However, as axial modulator 200 closes window 64, a lesser number of x-rays 206 are allowed to exit window 64. The remaining blocked x-rays 206 remain within needle 52. In another embodiment, referring to FIG. 13, a circumferential modulator 210 is illustrated wherein control cannula 202 is rotated about the central axis in order to selectively close window 64.

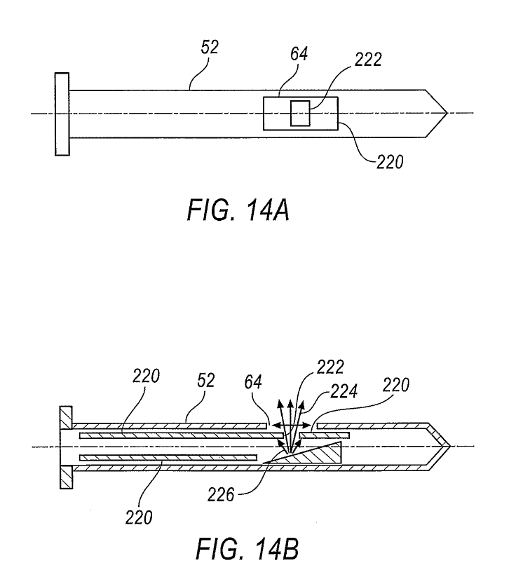

In some embodiments, referring to FIGS. 14A and 14B, a slot modulator 200 is shown. Slot modulator 220 includes a slot 222 and slides within needle 52. As slot modulator 200 is positioned relative to window 64, the pattern and position of emitted x-rays 224 may be controlled. At the same time, blocked x-rays 226 are contained within needle 52. Thus, the geometry of slot 222 may be selected to provide the desired x-ray pattern emitted from the device.

Referring to FIGS. 12-14, modulators 200, 210, and 220 maybe considered shutters that selectively modify the geometry of window 64 and allow or reject the flow of x-rays from interacting with patient 60. Each embodiment of modulators 200, 210, and 220 may be controlled axially or circumferentially with precision motors and actuators known to those skilled in the art.

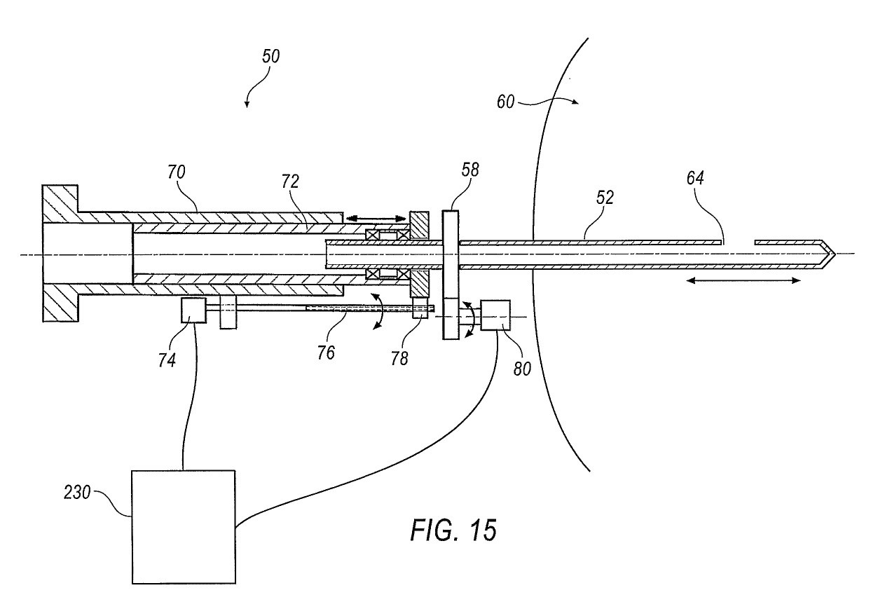

Referring to FIG. 15, a simplified view of positioning apparatus 50 is shown for positioning needle 52. As discussed with reference to FIG. 1, the depth of needle 52, and thus window 64, is controlled by axial motor 74. Also, the rotation of needle 52, and thus window 64, is controlled by rotary motor 80. Each motor 74, 80 may be of stepper motors controlled by a data processor 230. Thus, data processor 230 may control the position of window 64 within patient 60. Further, data processor 230 may control the position of modulators 200, 210, and 220 through actuators.

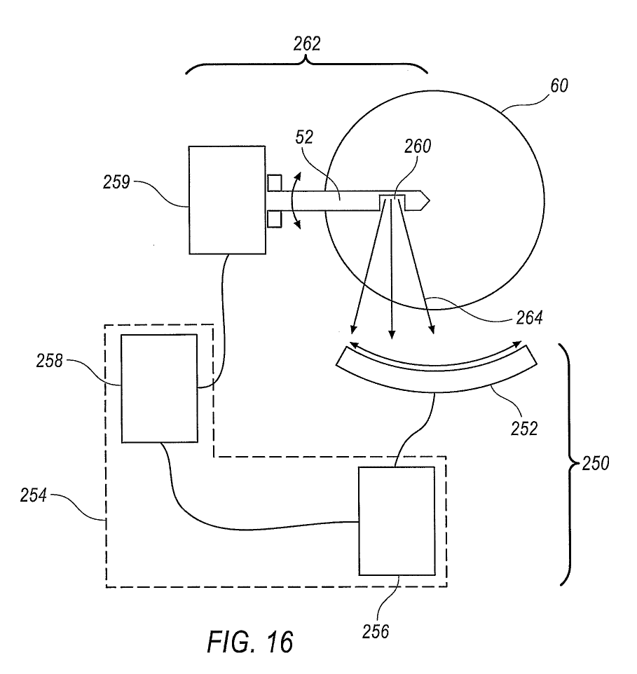

Referring to FIG. 16, an imaging system 250 for use in conjunction with system 40 is shown. Imaging system 250 comprises a detector 252 that is sensitive to x-rays, or low-energy photons. Detector 252 may be a simple slot-type detector or a multi-element array of x-ray sensitive regions. Detector 252 is operatively coupled to a processor 254, or data processor, that processes the output of detector 252. Further, processor 254 may further comprise an image processor 256 and a data processor 258. Additionally, processor 254 may be connected to a control system 259 that controls the location, direction, and intensity of an x-ray source 260. Thus, imaging system 250 may be used to guide the treatment procedure.

Using the imaging information provided by imaging system 250, image-guidance treatment techniques may be used where one or more internal x-ray sources 260 are generated with an insertable x-ray treatment device 262 coupled with either an analog or a digital detector 252. The x-ray sources produced in the treatment needle 52 may be used to image the treatment tissue and guide the treatment procedure including identifying treatment location 56, adjusting the in situ x-ray source (such as pseudo-target), location and orientation in the insertable device (such as needle), monitoring the treatment process, and documenting treatment, and other similar processes and procedures.

Without limitation, a function of imaging system 250 and processor 254 may be to ensure that the radiation device is providing a determinable amount of radiation at treatment location 56. The detector 252 and processor 254 may be configured to verify the accuracy of the treatment or imaging process and to initiate any necessary changes. As one example only, if the treatment device is not providing the desired amount or shape of radiation, processor 254 and detector 252 may either provide the treatment device with the necessary instructions to control system 259 to alter the amount and shape of the radiation or processor 254 may provide a signal to health care providers indicating that the amount and shape of radiation is not within desired ranges.

An automatic controlling mechanism may be used to process the delivery operation of optimized dose distribution with an insertable in situ x-ray brachytherapy device, such as needle 52. Alternatively, the controlling mechanism may be manually controlled. For example, the mechanism may be used to adjust the x-ray source (such as pseudo-target) location, orientation, geometry, intensity (radiation time), beam energy, and number of in situ x-ray sources and locations.

Further, both needle 52 and detector 252 may be rotated so that detector 252 may capture images of different angles of patient 60. In such an embodiment, processor 254 may control then positioning of both needle 52 and detector 252, as well as controlling the intensity of x-rays 264 emitted from x-ray sources 260. The method of controlling imaging system 250 as well as x-ray sources 260 is explained below with respect to tomographic imaging.

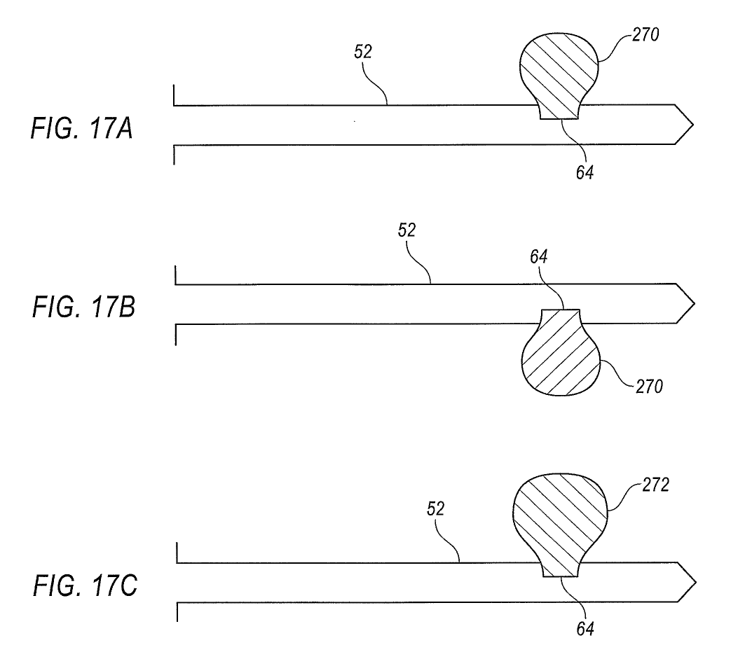

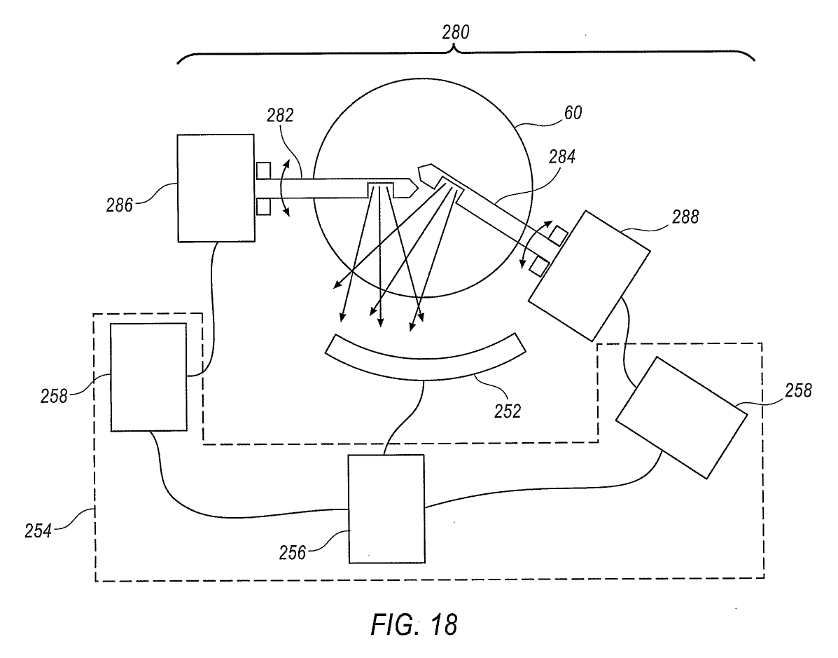

Referring to FIGS. 17A-17C, a simplified illustration of the rotational control and energy modulation of system 40 is shown. FIG. 17A shows a first position for needle 52 and window 64 having a first energy of x-rays 270. Control system 259 may then be commanded to rotate needle 52 to a second position while emitting the same first energy of x-rays 270, as is shown in FIG. 17B. However, control system 259 may also be commanded to return to the first position while increasing the x-ray energy to a second energy level 272. Control system 259 may adjust the x-ray source location laterally, and rotationally, as well as change the orientation, geometry, intensity, and x-ray energy emitted from system 40.

Referring to FIG. 18, a multiple source in situ system 280 is shown. A first needle 282 and a second needle 284 are both inserted into patient 60. Thus, multiple in situ x-ray sources may operate simultaneously, exclusively, or a combination thereof, to provide treatment. Similar to the system of FIG. 16, detector 252 receives x-rays exiting patient 60 and communicates the image information to image processor 256. However, processor 254 may then process the imaging information and control both first needle 282 and second needle 284 via control systems 286 and 288 respectively. In some embodiments, without limitation, multiple source in situ system 280 may include more than two x-ray sources, and may include a combination of modalities of sources, such as an externally generated x-ray source 100 (see FIG. 2), an in situ generated x-ray source 140 (see FIG. 6), and a radiative material source 170 (see FIG. 8). Additionally, each x-ray source may include a heating element 130 (see FIG. 5), or a heating element may be separately introduced into patient 60.

Generally, the x-ray source comprises an image guided insertable in situ treatment device. The device comprises an insertable in situ device (such as a needle, a probe, or similar type) with low-energy photon beams, with an external x-ray source 100 coupled with an x-ray transportation path to the in situ target 100, an x-ray generated in situ device 140, or a radiative pellet 170, a kV x-ray imaging device 252, a planning system that determines dose distributions and dose modulation in terms of their output, energy, orientation, and locations, a mechanism to control the above operations, and a mechanism to generate heat in the localized insertable in situ device. The insertable device has a mechanism to perform intensity modulation by moving the in situ x-ray source or changing the beam shaping devices.

The image-guided system may include a kV x-ray imaging device and an x-ray detector, either in digital or analog format. In the image-guided system, the treatment x-ray source may be used as the imaging x-ray source. A detector may be used to record the x-ray information passing through the treatment tissues.

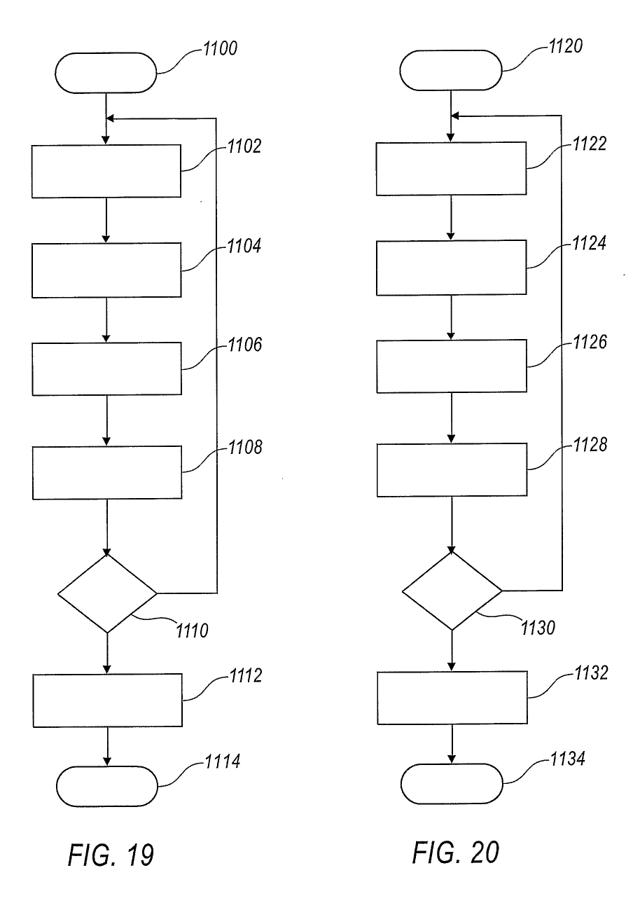

Referring to FIG. 19, an image guidance process is shown. In order to generate kV x-ray images in the treatment region to make sure the proper dose is delivered to the appropriate region. If not, the radiographic images could be used to adjust the source orientation and location during the treatment process. In step 1100, the process begins. Control proceeds to step 1102.

In step 1102, the control enables the x-ray emitter. Control proceeds to step 1104.

In step 1104, the process commands the x-ray source to produce x-rays at a user-determinable energy level in order to treat a region or merely to image the region for further consideration. Control proceeds to step 1106.

In step 1106, the imager, or detector, captures a frame of information related to the region subject to the x-ray source. Control proceeds to step 1108.

In step 1108, processor 259 determines the dosage present at the imaged region. Processor 259 may also be configured to provide an image of the region to a surgeon. Control proceeds to step 1110.

In step 1110, processor 259 decides whether to continue treating the region or to move to the next region of interest. If processor 259 determines that the region has been appropriately dosed, control proceeds to step 1112. Otherwise, control proceeds to step 1102 where additional dosing of the region is provided for.

In step 1112, processor continues with the next region of interest for treatment. The process ends following step 1112 at 1114.

Additionally, the imaging device may used to monitor the treatment process using fluoroscopic imaging method. For this process, the imaging detector may be a digital format and may sample at more than seven frames per second. This real-time imaging method tracks the radiation sources relative to the treatment region.

Referring to FIG. 20, in step 1120, the process begins. Control proceeds to step 1122.

In step 1122, the control enables the x-ray emitter. Control proceeds to step 1124.

In step 1124, the process commands the x-ray source to produce x-rays at a user-determinable energy level in order to treat a region or simply to image the region for further consideration. Control proceeds to step 1126.

In step 1126, the imager is configured to capture images and dosing information periodically. Typical capture rates include at least seven frames per second. Control proceeds to step 1128.

In step 1128, processor 259 determines the dosage present at the imaged region. Processor 259 may also be configured to provide an image of the region to a surgeon. Control proceeds to step 1130.

In step 1130, processor 259 decides whether to continue treating the region or to move to the next region of interest. If processor 259 determines that the region has been appropriately dosed, control proceeds to step 1132. Otherwise, control proceeds to step 1122 where additional dosing of the region is provided for.

In step 1132, processor continues with the next region of interest for treatment. The process ends following step 1132 at 1134.

Additionally, the imaging device maybe used to verify and document the treatment. Radiographic images may be used to document the treatment region for the purpose of treatment verification. Also, if the detector is exposed to the entire treatment, the cumulated information can be used to monitor the radiation dose and used to shut the radiation off when it is over the prescribed dose. For this application, the detector is calibrated prior the application.

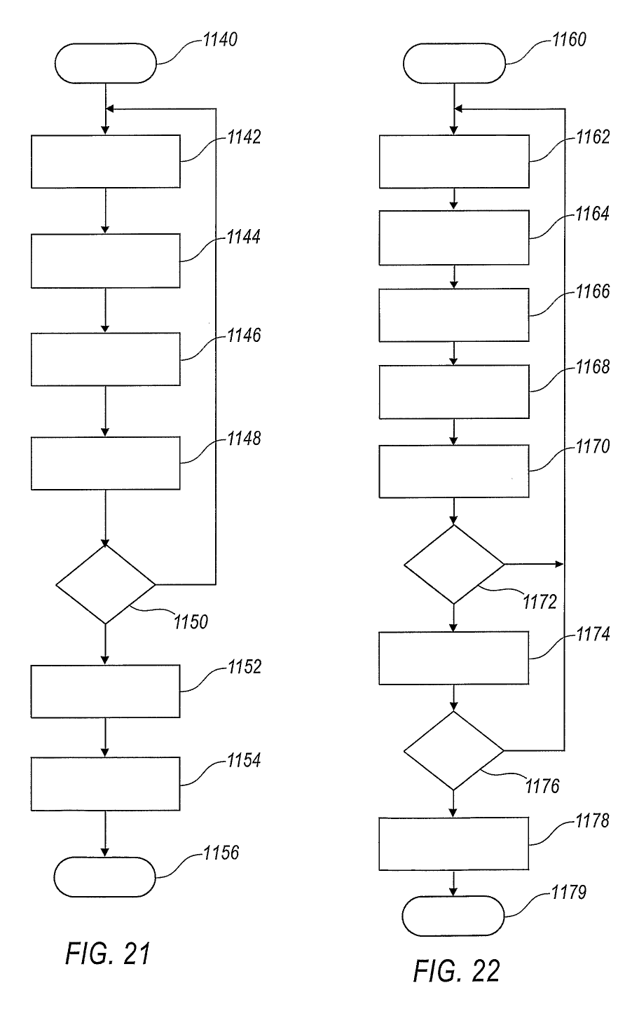

Referring to FIG. 21, in step 1140 the process begins. Control transfers to step 1142.

In step 1142, a predetermined dose threshold is determined for future use to discontinue treatment. The predetermined dose threshold may be programmed by a data processor or may be manually entered. Further, an upper end safety threshold may be used where the programmed dose threshold is abnormally high. Control proceeds to step 1144.

In step 1144, an x-ray emitter is enabled where x-rays may then interact with the patient. Control proceeds to step 1146.

In step 1146, an imager collects captures images and dosing information. Control proceeds to step 1148.

In step 1148, processor 254 archives the images and dosing information for future reference. Control proceeds to step 1150.

In step 1150, processor 254 determines whether the predetermined dose threshold has been met. If so, control proceeds to step 1152. If not, control proceeds to step 1144 where more exposure to x-rays is allowed.

In step 1152, processor 254 commands the x-ray source to stop emitting x-rays. Control proceeds to step 1154.

In step 1154, processor 254 presents the archived image and dosing information, stored in step 1148, to another data processor or a surgeon for further analysis or storage. The process ends after step 1154 at 1156.

Additionally, multiple projection images produced by the x-ray source produced in the in situ treatment device can be used to reconstruct tomographic images for 3-D visualization, treatment localization, treatment analysis and verification, and for other similar needs. In some embodiments, without limitation, the tomographic imaging system uses the image-guided system with the addition of a rotation mechanism for both x-ray source and the detector and an image reconstruction algorithm to generate a tomographic image. The x-ray source and the detector can rotate together so that, for each rotation angle, a projection image could be obtained. These projection images can be reconstructed as tomographic image so that treatment region can be viewed in three-dimensional (3-D) space. Also, this 3-D image can be used to guide the treatment process in a manner, similar to the previous 2-D projection images.

Referring to FIG. 22, a tomographic imaging and guidance procedure is outlined. In step 1160 the process begins. Control transfers to step 1162.

In step 1162, control system 259 positions and orients an x-ray emitter 260. Control system 259 may position x-ray emitter axially along the central axis of needle 52, or control system 259 may rotate x-ray emitter 260 (see FIG. 16). Control proceeds to step 1164.

In step 1164, control system 259 controls the intensity of x-ray source 260. In order to provide proper dosing to treatment location 56, and provide the appropriate x-ray intensity to enable imaging, control system 259 modulates the intensity of the emitted x-rays 260. Control proceeds to step 1166.

In step 1166, detector 252 is positioned relative to x-ray source 260. Control proceeds to step 1168.

In step 1168, detector 252 produces an image from said incident x-rays. Processor 254 uses or displays the image made in conjunction with the in situ therapy. As will be appreciated, a sub-procedure for control and dosing similar to those described in FIG. 19 may be used at this step. Control proceeds to step 1170.

In step 1170, the two dimensional image captured in step 1168 is stored for later analysis and construction into a three dimensional tomographic image. Control proceeds to step 1172.

In step 1172, processor 254 determines whether the appropriate number of two dimensional images have been collected in order to generate a three dimensional tomographic image. If an appropriate number of images have been stored, control proceeds to step 1174. If not, control proceeds to step 1162 for additional image collection and treatment.

In step 1174, a three dimensional tomographic image is constructed in the manner known to those skilled in the art. The three dimensional image is then displayed or used by processor 254 to further define and refine treatment. An optimizing algorithm, such as an inverse planning algorithm may be used to determine the most efficient treatment of treatment location 56 within the three dimensional space. Such algorithms may comprise a gradient optimization method, a fuzzy logic method, a simulated annealing method, and a method, to be explained below in detail. Control proceeds to step 1176.

In step 1176, processor 254 determines whether the treatment is complete. Based on the two and three dimensional images providing dosing information, processor 254 compares the treatment dosing and coverage to a predetermined treatment plan. If treatment is not complete, treatment continues as control is transferred to step 1162. If treatment is complete, control proceeds to step 1178.

In step 1178, processor 254 may store the completed treatment and imaging information for further analysis. The process ends after step 1178 at 1179.

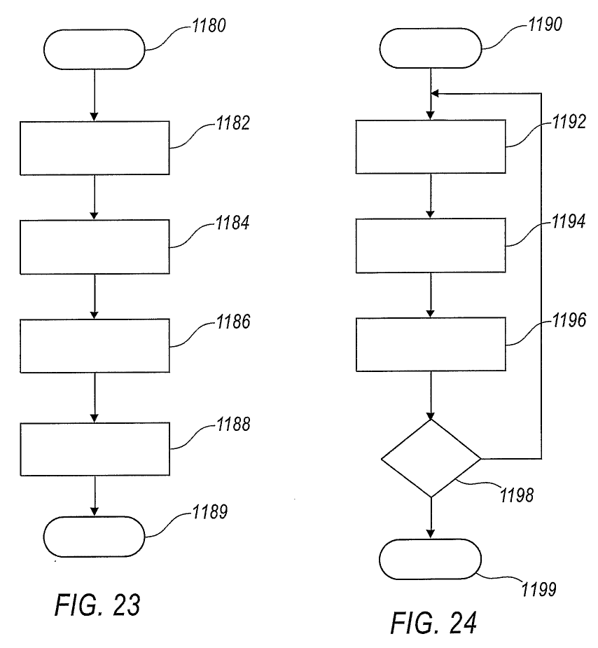

Generally, control system 259 may control every aspect of the nature of x-ray source 260 and needle 52 position and orientation (see FIGS. 1, 10-14, and 16). Referring to FIG. 23, in step 1180 the process begins. Control transfers to step 1182.

In step 1182, processor 254 determines the desired position and orientation of source 260 within patient 60. Processor 254 then commands the desired position to control system 259. Control transfers to step 1184.

In step 1184, control system 254 controls axial motor 74 to drive needle 52 in order to position source 260 (see FIGS. 1 and 16). Control transfers to step 1186.

In step 1186, control system 254 controls axial motor 74 to drive needle 52 in order to position source 260 (see FIGS. 1 and 16). Control transfers to step 1188.

In step 1188, control system 254 confirms the axial and rotational position of source 260. The process ends after step 1188 at 1189.

Additionally, the intensity and time of emission of the x-ray source may be modulated in order to provide precise control over the dosage applied to patient 60. Referring to FIG. 24, in step 1190 the process begins. Control transfers to step 1192.

In step 1192, processor 254 determines the type of treatment device that is being used in the procedure, such as externally generated x-ray source 100 (see FIG. 2), in situ generated x-ray source 140, and radiative material source 170. Additionally, each x-ray source may include a heating element 130, or a heating element may be separately introduced into patient 60. Given the type of treatment device chosen, the operating parameters are determined. Control transfers to step 1194.

In step 1194, processor 254 determines the dosage time and energy required for the present portion of the treatment based upon treatment location 56 and the treatment device parameters. Control proceeds to step 1196.

In step 1196, processor 254 commands control system 259 to enable the x-ray source for the dosage time and energy determined in step 1194. Control system 259 then modulates the x-ray source to provide the commanded dosage time and energy. Where source 260 is a radiative material source 170, control system 259 may not be able to control the energy level of the x-rays emitted from radiative material source 170. However, control system 259 can control the exposure time by modulating shutter 200. Control proceeds to step 1198.

In step 1198, processor 254 determines if more dosing is required. If so, control proceeds to step 1192. If not, the process ends after step 1198 at 1199.

One of ordinary skill in the art will appreciate that the positioning algorithms and dosing algorithms may be used in conjunction with each other. Also, one of ordinary skill in the art will appreciate that modulation of the time and energy level of generated x-ray sources can be applied to a multitude of sourcing modalities.

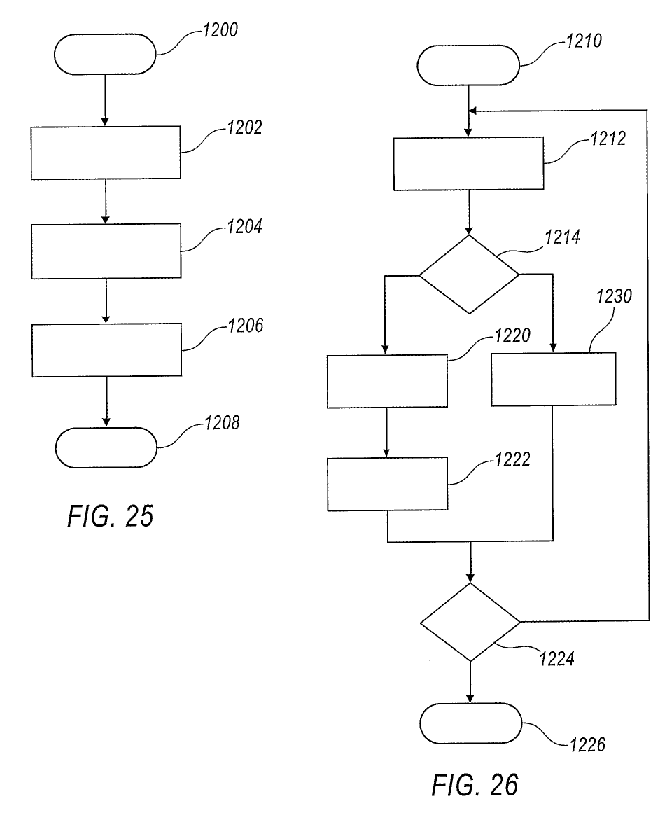

Alternately, without limiting the scope of the invention, time and shape of the x-ray source may be modulated using the mechanical methods based on controlling a shutter 200 that selectively obstructs window 64 (see FIGS. 10-14). Referring to FIG. 25, in step 1200 the process begins. Control proceeds to step 1202.

In step 1202, processor 254 determines the x-ray source modality and the desired x-ray dispersion pattern. Control proceeds to step 1204.

In step 1204, processor 254 determines the optimal shutter position in order to achieve the desired x-ray dispersion pattern based on the geometry of window 62 and the geometry of shutter 200. Alternately, a multi window needle 52 may be used along with a specialized geometry of shutter 200. Processor 254 then commands control system 259 with the desired shutter position. Control proceeds to step 1206.

In step 1206, control device 259 commands actuators to move shutter 200 axially and rotationally relative to window 64. The process ends after step 1206 at 1208.

A heating device may also require control depending upon the type of device. However, more sophisticated algorithms may be required where the heating device is passive and is heated by the x-ray source in situ. Such algorithms are within the skill and knowledge of those of ordinary skill in the art. Referring to FIG. 26, in step 1210 the process begins. Control transfers to step 1212.

In step 1212, processor 254 determines whether an adjunctive treatment, such as a heating device, should be engaged for the present phase of treatment. If adjunctive therapy is required, control proceeds to step 1214. Otherwise, control remains at step 1212.

In step 1214, processor 254 determines whether the adjunctive device is actively controlled or passively controlled. If the adjunctive device is actively controlled, control transfers to step 1220. If the adjunctive deice is passive, control transfer to step 1230.

In step 1220, processor 254 commands control system 259 to modulate the heat output by the adjunctive device. Control then passes to step 1222.

In step 1222, processor 254 records the time, duration, location, and intensity of the adjunctive therapy for further processing. Control then passes to step 1224.

In step 1230, the passive adjunctive device is heated by the in situ x-rays. Processor 254 records the time, duration, location, and intensity of the adjunctive therapy for further processing. Further, processor 254 is aware of the nature, location, and characteristics of the adjunctive device. Therefore, direct control of the adjunctive device may not be necessary. However, processor 254 and control system 259 may indirectly control the heating by controlling the emission of x-rays from the in situ source. Control then passes to step 1224.

In step 1224, processor 254 determines whether continued adjunctive therapy is required. If so, control is transferred to step 1212. If continued adjunctive therapy is not required, the process ends after step 1224 at 1226.

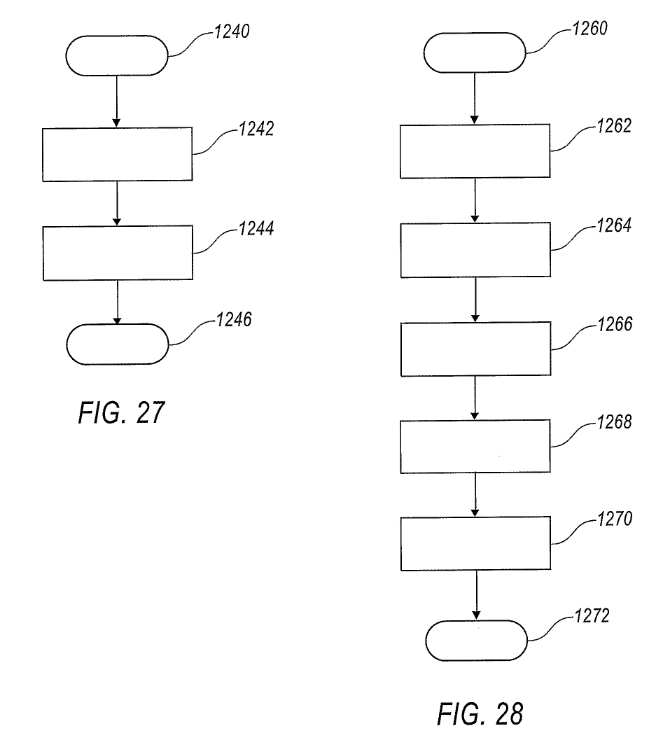

Mapping of treatment progress is also important to determine the extent and location of dosing provided to patient 60. Referring to FIG. 27, in step 1240 the process begins. Control transfers to step 1242.

In step 1242, the desired mapping and dosing information is loaded by processor 254. The actual mapping and dosing is loaded from stored imaging and detecting passes from the treatment procedure. Control is passed to step 1244.

In step 1244, the desired and actual mapping and dosing information is compared to determine whether treatment location 56 was dosed appropriately, and if so, was a margin applied to the outer limits to provide a safety zone. Such a comparison of the treatment information is useful in all decision stages of all algorithms described herein. The process ends after step 1244 at 1246.

The treatment planning system determines dose distributions and dose modulation in terms of their output, energy, orientation, and locations. A mechanism is included to control these operations. In some embodiments, without limitation, also included may be a mechanism to generate heat in the localized insertable in situ device. The planned results can be delivered by the insertable device, which has a control system to perform intensity modulation by moving the in situ x-ray source or by changing the beam-shaping devices.

An automatic controlling mechanism can be used to process the delivery operation of optimized dose distribution with an insertable in situ x-ray brachytherapy device (such as needle-based). Alternatively, the controlling mechanism can be manually controlled. For example, the mechanism can be used to adjust the x-ray source (such as pseudo-target) location, orientation, geometry, and intensity (radiation time), beam energy, and number of in situ x-ray sources (such as needle number) and locations.

An inverse planning method (such as gradient optimization method, fuzzy logic method, simulated annealing method, method, etc) can be used to optimize dose distribution for an insertable in situ x-ray treatment system for intensity-modulated brachytherapy. The method further comprises optimization of in situ x-ray source (such as pseudo-target in citation 1) location, orientation, geometry, intensity (radiation time), beam energy, and number of in situ x-ray sources (such as needle number) and locations.

Referring to FIG. 28, in step 1260 the process begins. Control transfers to step 1262.

In step 1262, processor 254 defines a treatment envelope using in situ imaging or prior imaging techniques that are correlated to the present imaged treatment location 56. Control passes to step 1264.

In step 1264, processor 254 determines the nature and type of the treatment device, including adjunctive treatment devices. The precise parameters of operation for the treatment device are loaded into processor 254. Control is passed to step 1266.

In step 1266, processor 254 determines a performance envelope based on the type of treatment device selected and the imaged treatment location 56. The performance envelope and treatment envelope are compared to initially determine the outer limits of the treatment plan. The treatment plan is determined from the possible locations, orientations, geometry, intensities, beam energies, and number of in situ x-ray sources and their respective locations. Control is passed to step 1268.

In step 1268, processor 254 applies an optimization algorithm to the treatment plan to determine the most efficient plan of exposing treatment location 56 to the x-rays. The inverse planning algorithm optimization may be a gradient optimization method, fuzzy logic method, simulated annealing method, or method. Those skilled in the art of rapid optimization for complex problems will appreciate that any method, not only those mentioned, may be utilized for the inverse planning algoritlun. In general, the algorithms operate on a complex set in order to locate the global maximum and minimum while using less computational time than an exhaustive approach. Control is passed to step 1270.

In step 1270, processor 254 applies the optimized treatment plan to the in situ system and automatically executes the treatment plan. To those skilled in the art, the aforementioned embodiments of processor 254, control system 259, and imaging system 250 may be used to execute the results of treatment planning by automatically positioning the x-ray source location and orientation, and controlling the radiation output. The process ends after step 1270 at 1272.

The present invention has been particularly shown and described with reference to the foregoing embodiments, which are merely illustrative of the best modes for carrying out the invention. It should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. Moreover, the foregoing embodiments are illustrative, and no single feature or element is essential to all possible combinations that may be claimed in this or a later application.

1. A modulated image-guided x-ray brachytherapy system comprising:
a source providing low-energy photons, said source configured for placement at least partially within a patient;
a control system configured to modulate said source; and
an imaging system for locating said source relative to a treatment location in a patient.

2. The system of claim 1, said system further comprising:
a target located at least partially within the patient;
a generator located outside the patient for providing low-energy photons, said generator configured to direct low-energy photons to said target; and
a conduit between said generator and said target providing a path for said low-energy photons to travel inside the patient.

3. The system of claim 2, wherein said target is metallic.

4. The system of claim 2, wherein said target is crystalline.

5. The system of claim 2, wherein said target is selectively movable relative to said conduit.

6. The system of claim 5, wherein said target is rotably mounted to said conduit, whereby rotation of said target modulates the intensity of the low-energy photons.

7. The system of claim 2, further comprising:
a mount engaged with said conduit and said target, said mount surrounding said target; and
at least one opening disposed upon said mount, whereby said at least one opening provides a path for the low-energy photons to interact with the patient.

8. The system of claim 7, further comprising:
a shutter for selectively obstructing said at least one opening.

9. The system of claim 2, wherein said control system selectively modulates the intensity of the low-energy photons provided by said generator.

10. The system of claim 1, said source further comprising:
an in situ x-ray generator for insertion within the patient.

11. The system of claim 10, wherein the control system modulates the intensity of the x-rays produced by said in situ x-ray generator.

12. The system of claim 10, further including:
a casing at least partially surrounding said in situ x-ray generator.

13. The system of claim 12, wherein said casing further comprises:
at least one opening disposed upon said casing, said opening providing a path for the x-rays to interact with the patient.

14. The system of claim 13, further including:
a shutter for selectively obstructing at least a portion of said at least one opening.

15. The system of claim 1, wherein said source is a radioactive pellet.

16. The system of claim 15, further including:
a casing at least partially surrounding said radioactive pellet.

17. The system of claim 16, wherein said control system selectively positions said radioactive pellet relative to said casing.

18. The system of claim 15, further including:
at least one opening disposed upon said casing.

19. The system of claim 18, further including:
a shutter for selectively obstructing said at least one opening, said control system selectively positioning; said shutter relative to said opening.

20. An image-guided intensity-modulated x-ray brachytherapy system comprising:
a source providing low-energy photons for treatment of a patient, the source configured for placement at least partially within a patient;
a control element for modulating the intensity of the source;
a detector sensitive to the low-energy photons; and
a processor operatively coupled to said detector and said control element, said processor producing an image of a treatment location.

21. The system of claim 20, wherein said processor is operatively coupled to said source.

22. The system of claim 20, wherein said image guides the treatment.

23. The system of claim 20, wherein said image identifies the treatment location.

24. The system of claim 20, wherein said image provides information for adjusting said source location within the patient.

25. The system of claim 20, wherein said image provides information for adjusting a source orientation within the patient.

26. The system of claim 20, wherein said image provides information for adjusting said control element.

27. The system of claim 20, wherein said image provides information for monitoring the treatment process.

28. The system of claim 20, wherein said image provides information for documenting the treatment process.

29. The system of claim 20, wherein the detector is digital.

30. The system of claim 20, wherein the detector is analog.

31. An image-guided intensity-modulated x-ray brachytherapy system comprising:
a rotable x-ray source configured for placement at least partially within a patient;
an x-ray detector having a plurality of sensing elements for detecting x-rays that have passed through the patient; and
a processor operatively connected to said x-ray detector for processing a plurality of output images from said x-ray detector to produce a tomographic image, whereby the tomographic image is used for at least one of a three dimensional visualization of a treatment location, treatment localization, treatment analysis, and treatment verification.

32. The system of claim 31, further comprising:
an external x-ray generator, said external x-ray generator positioned outside said patient; and
wherein said rotable x-ray source comprises a target for redirecting the x-rays provided by said external x-ray generator.

33. The system of claim 31, wherein said rotable x-ray source comprises an in situ x-ray generator.

34. The system of claim 31, wherein said rotable x-ray source comprises a radioactive pellet.

35. An image-guided intensity-modulated x-ray brachytherapy system comprising:
a rotable x-ray source configured for in situ treatment within a patient;
an x-ray detector having a plurality of sensing elements and producing an output; and
a processor operatively connected to said x-ray detector for storing a plurality of outputs, said processor constructing a three dimensional representation of a treatment location within the patient.

36. The system of claim 35, wherein the three dimensional representation provides visualization of the treatment location.

37. The system of claim 35, wherein the three dimensional representation provides treatment localization.

38. The system of claim 35, wherein the three dimensional representation provides information for analyzing the treatment.

39. The system of claim 35, wherein the three dimensional representation provides information for treatment verification.

40. A system for image-guided intensity-modulated x-ray brachytherapy comprising:
at least one x-ray source configured for placement at least partially within a patient;
a control system modulating said at least one x-ray source;
a data processor operatively connected to said control system; and
wherein said data processor determines a strategy for at least one x-ray source location and at least one x-ray source intensity for treating at least one treatment location; and
wherein said data processor communicates said strategy to said control system.

41. The system of claim 40, said strategy further including:
an optimizing algorithm for improving the efficiency of locating the at least one source and modulating the at least one x-ray source.

42. The system of claim 41, wherein said optimizing algorithm is an inverse planning algorithm.

43. The system of claim 41, wherein said optimizing algorithm comprises at least one of one of a gradient optimization method, fuzzy logic method, simulated annealing method, and genetic method.

44. The system of claim 40, further comprising:
an imaging system operatively connected to said data processor providing guidance to said data processor for improving said strategy.

45. The system of claim 44, wherein said imaging system provides real-time guidance to said data processor.

46. A planning method for image-guided intensity-modulated x-ray brachytherapy comprising the steps of:
generating x-rays from at least one in situ x-ray source;
imaging a treatment location;
generating a treatment plan; and
modulating said at least one in situ x-ray source according to said treatment plan.

47. The method of claim 46, wherein said imaging utilizes said x-rays provided by said at least one in situ x-ray source.

48. The method of claim 46, wherein said imaging is a tomographic image.

49. The method of claim 46, wherein said treatment plan is generated using an optimizing algorithm.

50. The method of claim 50, wherein said optimizing algorithm is an inverse planning algorithm.

51. The method of claim 46, wherein said optimizing algorithm comprises at least one of one of a gradient optimization method, fuzzy logic method, simulated annealing method, and genetic method.

52. A system for controlling an image-guided intensity-modulated x-ray brachytherapy system comprising:
an in situ x-ray source located at least partially within a patient for treating a treatment location;
a modulator selectively controlling said in situ x-ray source intensity;
a positioner for locating said in situ x-ray source within a patient;
an imaging system for collecting real-time treatment information; and
a data processor operatively connected to said imaging system, said positioner, and said modulator; said data processor following a treatment plan; said data processor validating said treatment at said treatment location with said real-time treatment information; such that said data processor communicates with said positioner and said modulator to realize said treatment plan.

53. The system of claim 52, wherein said modulator controls at least one of a source energy level, a source density, a source shape, and a source angle of emission.

54. The system of claim 52, wherein locating said in situ x-ray source comprises at least one of a rotation angle and a depth of penetration within said patient.

55. The system of claim 52, further including:
a heating element located at least partially within a patient for treating the treatment location.

56. The system of claim 55, wherein said modulator further controls said heating element.

57. A method for controlling an image-guided intensity-modulated x-ray brachytherapy system comprising the steps of:
imaging a treatment location;
determining a method of treatment;
planning a radiative treatment cycle, said planning including an inverse treatment method to determine optimal exposures for the treatment location;
outputting a treatment plan; and
controlling a brachytherapy device using said treatment plan.

58. The method of claim 57, wherein said radiative treatment cycle comprises at least one component of time, location, radiative shape, radiative type.

59. The method of claim 57, wherein said output is used by a doctor to guide treatment.

60. The method of claim 57, wherein said output is used by a control system to implement the treatment plan.

61. The method of claim 57, wherein said brachytherapy device provides x-rays used in imaging the treatment location.

62. The method of claim 57, wherein said inverse planning method comprises at least one of a gradient optimization method, fuzzy logic method, simulated annealing method, and genetic method.

63. The method of claim 57, wherein controlling said brachytherapy device is automatic.

64. The method of claim 57, wherein the inverse planning method is updated based upon a dosing projection provided by the imaging.

65. The method of claim 57, wherein imaging the treatment location is performed in real-time during treatment.

66. The method of claim 64, wherein the inverse planning method is updated based upon a dosing projection provided by the real-time imaging.

67. The method of claim 57, wherein the step of determining a method of treatment further comprises considering use of an adjunctive treatment.

68. The method of claim 67, wherein the adjunctive treatment is heat.

69. An image-guided intensity-modulated x-ray brachytherapy system comprising:
a probe configured for insertion at least partially within a patient;
an x-ray source configured for placement at least partially within a patient; and
an adjunctive therapy for treating the patient;

70. The system of claim 70, wherein the adjunctive therapy comprises heating a portion of the probe.
